(19) United States
(12) Patent Application Publication
(10) Pub. No.: US 2009/0215774 A1
(43) Pub. Date: Aug. 27, 2009

(54) USES OF INDANE COMPOUNDS

(75) Inventors: Jan Kehler, Kgs. Lyngby (DK); Karsten Juhl, Greve (DK); Ask Puschl, Frederiksberg (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. LUNDBECK A/S, Valby-Copenhagen (DK)

(21) Appl. No.: 12/390,473

(22) Filed: Feb. 22, 2009

Related U.S. Application Data
(60) Provisional application No. 61/031,459, filed on Feb. 26, 2008.
(60) Provisional application No. 61/031,820, filed on Feb. 27, 2008.

(51) Int. Cl. A61K 31/5377; A61K 31/451; A61K 31/404; A61P 3/04

(52) U.S. Cl. 514/235.2; 514/323; 514/415

(57) ABSTRACT

The invention provides methods of treating obesity and eating disorders, which comprise administering indane compounds of Formula (I):

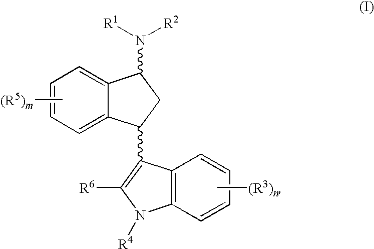

where R¹-R⁶, m and n are defined herein.

### CROSS-REFERENCE TO RELATED APPLICATIONS

This application is a U.S. nonprovisional patent application that claims the benefit of U.S. provisional patent applications, U.S. Ser. No. 61/031,459, filed Feb. 26, 2008, and U.S. Ser. No. 61/031,820, filed Feb. 27, 2008, each of which are incorporated by reference in its entirety.

### FIELD OF THE INVENTION

The invention provides methods of treating obesity and eating disorders, which comprise administering indane compounds of Formula I.

### BACKGROUND OF THE INVENTION

There are compounds with a triple monoamine reuptake mechanism, in different phases of preclinical or clinical trials for obesity, including Tesofensine, Sibutramine, and DOV21947. The triple uptake inhibitors (SSRI/SNRI/selective dopamine re-uptake inhibitor) inhibit the uptake of all three neurotransmitters that are linked to depression: serotonin, noradrenaline and dopamine.

Sibutramine is a satiety-inducing serotonin-noradrenaline re-uptake inhibitor (SNRI) that acts predominantly via its primary and secondary metabolites (has two active metabolites, desmethylsibutramine and didesmethylsibutramine; M1 and M2). These metabolites are more potent at inhibiting uptake of norepinephrine and dopamine than of serotonin (in vitro) (Glick, S. D., et al.). The effect of Sibutramine on energy expenditure in rats is predominantly due to a dopamine-dependent increase in locomotor activity (Golozoubova, V., et al.). Sibutramine's anti-obesity action is probably attributable to effects on energy intake and expenditure (Liu, Y. L., et al., EJP 2002), although a reduction of food intake seems to play a role. Coletta, D. K., et al. reported preliminary results in mice suggesting that the M2 metabolite of Sibutramine can reduce glycaemia, maintain insulin-mediated muscle glucose uptake and reduce hepatic gluconeogenesis independently of satiety and weight loss. Richardson, D. K., et al. indicate that the Sibutramine metabolite M2 can act directly on murine and human adipose tissue to increase lipolysis via a pathway involving beta-adrenoceptors. In addition, Liu, Y. L., et al., IJORMD, 2002) reported that Sibutramine's metabolite M2 increased 5-HT and NE tone via potent reuptake inhibition, which subsequently results in increased efferent sympathetic activity to brown adipose tissue (BAT).

Tesofensine (NS 2330) is a novel triple monoamine reuptake inhibitor that finished clinical phase 2b for obesity. Data from the study in 203 patients show that 24 weeks' treatment with 0.25 mg, 0.5 mg and 1 mg Tesofensine resulted in a dose-dependent average weight loss of 6.5%, 11.2% and 12.6%, respectively and also affected BMI (Body Mass Index (kg/m2)), as well as feeling of satiety and appetite (Neurosearch.com).

DOV 21947 (preclinical development for obesity) inhibits NE, 5-HT and DA uptake, and reduces body weight (1-24 d treatment) in rodent models of diet-induced obesity (DIO). It caused a sustained, but reversible reduction in the rate of body weight gain for as long as 6 months in normal rats, and up to 1 year in normal dogs. The decrease in body weight resulted specifically from a loss of retroperitoneal and mesenteric depots of white adipose tissue. DOV 21947 also reduced daily food intake in DIO rats, but consumption returned to control levels after 11 days of treatment. DOV 21947 is effective in causing a sustained and selective reduction in fat content and triglyceride levels in animal models of obesity without significantly altering vital organ function (Tizzano, J. P. et al.).

Antidepressant medications, including tri-cyclic antidepressants, selective serotonin re-uptake inhibitors, as well as some of the novel antidepressants, have shown evidence of some therapeutic value in both Bulimia Nervosa (BN) and Binge Eating Disorder (BED), but not Anorexia Nervosa (AN) (Steffen, K. J., et al.). As far as the monoamine reuptake inhibitors, the NERI, SSRI, and SNRI effects on Obesity and Eating Disorders are established. The Norepinephrine re-uptake inhibitors Atomoxetine and Reboxetine are associated with weight loss and were efficacious in the short-term treatment of clinical Binge Eating Disorder (BED) (McElroy, S. L., et al.; Silveira, R. O., et al.). Duloxetine (SNRI) decreased feeding and has effects on energy intake and expenditure. Citalopram (SSRI) may be useful in depressed patients with Bulimia, whereas Fluoxetine (SSRI) is more specific for those with introjected Anger and Bulimia (Leombruni, P., et al.). Venlafaxine and Duloxetine, dual NE/5-HT reuptake inhibitors, significantly decreased acute food intake in freely-feeding rats. Neither Fluoxetine (SSRI) nor Nisoxetine (NERI) had significant effects on food intake during the 8 hours dark period. However, a combination of Fluoxetine and Nisoxetine significantly decreased food intake 2 and 8 hours after drug administration. This demonstrates that inhibition of 5-HT and Noradrenaline re-uptake can be an effective approach for the treatment of obesity (Jackson, H. C., et al.). Buproprion is on the market as a DA/NE reuptake inhibitor; this specific combination was not effective in preclinical feeding so Liu, Y. L., et al. notes that any potential weight-reducing effect of Bupropion is therefore likely due to thermogenesis.

The present invention provides novel uses of indane compounds which are inhibitors of serotonin, norepinephrine and dopamine re-uptake. The compounds of the invention are therefore useful in the treatment of obesity and eating disorders.

### REFERENCES

The following references and all other references cited throughout the specification are hereby incorporated by reference in their entirety.

Glick, S. D., et al. “Enantioselective behavioral effects of sibutramine metabolites”, Eur. J. Pharmacol. 2000 (May 26), 397(1):93-102.

Golozoubova, V., et al. “Locomotion is the major determinant of sibutramine-induced increase in energy expenditure”, Pharmacol Biochem Behavior, 2006 (April), 83(4):517-27 [Epub 2006 May 2].

Liu, Y. L., et al. “Comparison of the thermogenic and hypophagic effects of sibutramine's metabolite 2 and other monoamine reuptake inhibitors”, Eur. J. Pharmacol. 2002 (Sep. 27), 452(1):49-56.

Coletta, D. K., et al. “The sibutramine metabolite M2 improves muscle glucose uptake and reduces hepatic glucose output: preliminary data”, Diab Vasc Dis Res, 2006 (December), 3(3):186-8.

Richardson, D. K., et al. “The primary amine metabolite of sibutramine stimulates lipolysis in adipocytes isolated from lean and obese mice and in isolated human adipocytes”, Horm Metab Res, 2006 (November), 38(11):727-31.

Liu, Y. L., et al. “Mechanism of the thermogenic effect of Metabolite 2 (BTS 54 505), a major pharmacologically active metabolite of the novel anti-obesity drug, sibutramine”, Int J Obes Relat Metab Disord, 2002 (September), 26(9):1245-53.

Tizzano, J. P., et al. “The Triple Uptake Inhibitor DOV 21947 Reduces Body Weight and Plasma Triglycerides in Rodent Models of Diet-Induced Obesity”, J Pharmacol Exp Ther, 2007 (December), 18 [Epub ahead of print].

Steffen, K. J., et al. “Emerging drugs for eating disorder treatment”, Expert Opin. Emerg Drugs, 2006 (May), 11(2):315-36.

McElroy, S. L., et al. “Atomoxetine in the treatment of binge-eating disorder: a randomized placebo-controlled trial”, J Clin Psychiatry, 2007 (March), 68(3):390-8.

Silveira, R. O., et al. “An open trial of reboxetine in obese patients with binge eating disorder”, Eat Weight Disord, 2005 (December), 10(4):e93-6.

Hauser, R. A., et al. “Randomized trial of the triple monoamine reuptake inhibitor NS 2330 (tesofensine) in early Parkinson's disease”, Movement Disorders, 2007 (Feb. 15), 22(3):359-65.

Leombruni, P., et al. “Citalopram versus fluoxetine for the treatment of patients with bulimia nervosa: a single-blind randomized controlled trial”, Adv Ther, 2006 (May-June), 23(3):481-94.

Jackson, H. C., et al. “Comparison of the effects of sibutramine and other monoamine re-uptake inhibitors on food intake in the rat”, Br. J. Pharmacol, 1997 (August), 121(8):1758-62.

Liu, Y. L., et al. “Comparison of the thermogenic and hypophagic effects of sibutramine's metabolite 2 and other monoamine reuptake inhibitors” Eur J Pharmacol, 2002 (Sep. 27), 452(1):49-56.

### SUMMARY OF THE INVENTION

An object of the invention is to provide methods of treating obesity and eating disorders, comprising administering to a patient in need thereof a therapeutically effective amount of compounds of the Formulas I-X, as defined below, which are inhibitors of serotonin, norepinephrine and dopamine re-uptake.

In one embodiment of the present methods, the compounds of the Formulas I-X, as defined below, are useful in the treatment of obesity. In another embodiment of the present methods, the compounds of the Formulas I-X, as defined below, are useful in the treatment of eating disorders. To further illustrate but without limiting the invention, the eating disorder to be treated is selected from the group consisting of bulimia nervosa, anorexia nervosa and binge eating disorder.

In a second aspect, the present invention relates to the use of a compound of the Formulas I-X, as defined below, for the manufacture of a medicament useful for the treatment of obesity and eating disorders.

In one embodiment, the present invention relates to the use of a compound of the Formulas I-X, as defined below, for the manufacture of a medicament useful for the treatment of obesity. In another embodiment, the present invention relates to the use of a compound of the Formulas I-X, as defined below, for the manufacture of a medicament useful for the treatment of eating disorders. To further illustrate without limiting the invention, the eating disorder to be treated is selected from the group consisting of bulimia nervosa, anorexia nervosa and binge eating disorder.

In a third aspect, the present invention relates to the use of a pharmaceutical composition comprising a compound of Formulas I-X, as defined below, in a therapeutically effective amount together with one or more pharmaceutically acceptable carriers or diluents for the treatment of obesity and eating disorders.

In a fourth aspect, the present invention relates to treating a disease, where the inhibition of serotonin, and/or norepinephrine and/or dopamine re-uptake is implicated, comprising administration of a therapeutically effective amount of a compound of the Formulas I-X, as defined below, to a mammal including humans.

In a fifth aspect, the present invention relates to a method of treating obesity and eating disorders, comprising the administration of a therapeutically effective amount of a compound of the Formula I-X, as defined below, to a mammal including humans.

In one embodiment, the present invention relates to a method of treating obesity, comprising the administration of a therapeutically effective amount of a compound of the Formula I-X, as defined below, to a mammal including humans. In another embodiment, the present invention relates to a method of treating eating disorders, comprising the administration of a therapeutically effective amount of a compound of the Formula I-X, as defined below, to a mammal including humans. To further illustrate without limiting the invention, the eating disorder to be treated is selected from the group consisting of bulimia nervosa, anorexia nervosa and binge eating disorder.

In one embodiment, the present invention relates to a method of treating obesity, comprising administering to a patient in need thereof a therapeutically effective amount of a compound of the Formula I-X, as defined below. In another embodiment, the present invention relates to a method of treating an eating disorder, comprising administering to a patient in need thereof a therapeutically effective amount of a compound of the Formula I-X, as defined below. To further illustrate without limiting the invention, the eating disorder to be treated is selected from the group consisting of bulimia nervosa, anorexia nervosa and binge eating disorder.

### BRIEF DESCRIPTION OF THE DRAWINGS

FIG. 1 is a graphical representation of the effect of a compound of the present invention on body weight in an embodiment of present invention;

FIG. 2 is a graphical representation of the effect of a compound of the present invention on daily food intake in an embodiment of present invention; and

FIGS. 3a-c are a graphical representations of the effect of a compound of the present invention on body fat content in an embodiment of present invention.

### DETAILED DESCRIPTION OF THE INVENTION

The present invention provides a method of treating obesity or an eating disorder, comprising administering to a patient in need thereof a therapeutically effective amount of a compound represented by the Formula I:

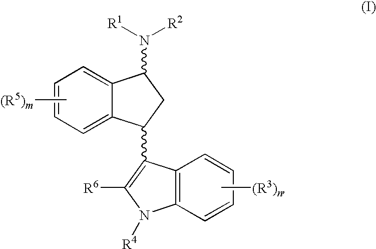

wherein:
each R¹ and R² are independently hydrogen, C₁-C₈-straight or branched alkyl or C₃-C₈-cycloalkyl; or wherein R¹ and R² and the nitrogen to which they are attached form azetidine, piperidine, pyrrolidine, azapane or morpholine;
each R³ is independently hydrogen, C₁-C₈-straight or branched alkyl, C₁-C₅-alkoxy, C₁-C₈-straight or branched polyfluoroalkyl, halogen, cyano, hydroxyl, tetrazole-optionally substituted with methyl, or amino; or wherein two R³ groups on adjacent carbons combine together to form a methylenedioxy linker;
R⁴ is hydrogen, C₁-C₈-straight or branched alkyl or C₃-C₈-cycloalkyl;
each R⁵ is hydrogen, halogen, C₁-C₅-alkoxy, C₁-C₈-straight or branched alkyl, C₁-C₈-straight or branched polyfluoroalkyl, cyano, or hydroxyl;
R⁶ is hydrogen, C₁-C₈-straight or branched alkyl or phenyl;
m is an integer from 1 to 4 inclusive; and
n is an integer from 1 to 4 inclusive;
   or pharmaceutically acceptable salts thereof.

In one embodiment, the methods of the present invention comprise compounds represented by the Formula I, which are the pure enantiomers, diasteromers and mixtures thereof.

In another embodiment, the methods of the present invention comprise a compound represented by the Formula I, which is the cis isomer of Formula II and III. In another embodiment, the methods of the present invention comprise a compound represented by the Formula I, which is the trans isomer of Formula IV and V.

In another embodiment, the present invention relates to a method of treating obesity or an eating disorder, wherein compounds of the Formula II-V are selected from the group consisting of:

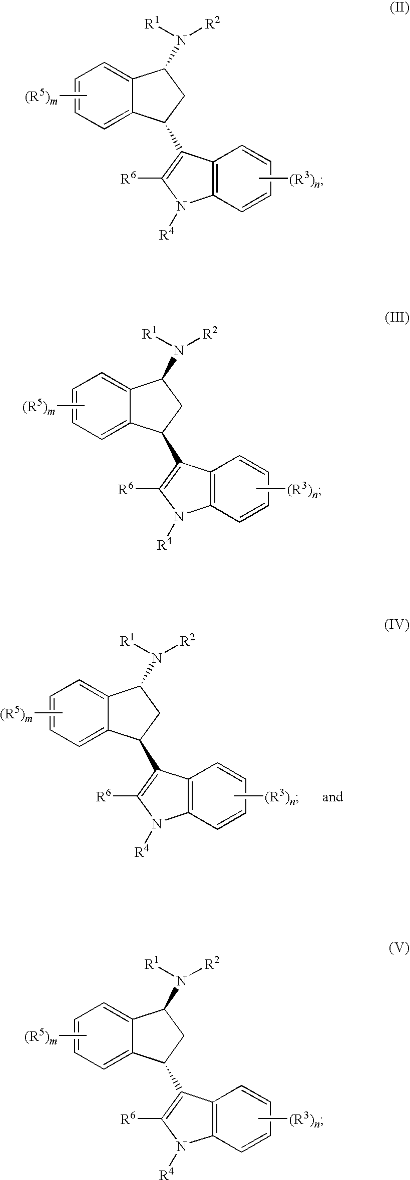

wherein R¹, R², R³, R⁴, R⁵, R⁶, m and n are as defined above.

In one embodiment of the present methods, R¹ is hydrogen. In one embodiment of the present methods, R² is methyl. In one embodiment of the present methods, R⁴ is hydrogen. In one embodiment of the present methods, R³ is selected form the group consisting of hydrogen, halogen, and methoxy. In one embodiment of the present methods, R³ is a hydrogen or halogen. In one embodiment of the present methods, the halogen is fluorine or chlorine. In one embodiment the present methods, R⁵ and R⁶ are hydrogen.

In another embodiment of the present methods, R¹ is hydrogen and R², R³, R⁴, R⁵, and R⁶ are as defined above in Formulas II-V. In a further embodiment of the present methods, R¹ is hydrogen, R² is methyl, R³, R⁴, R⁵ and R⁶ are as defined above in Formulas II-V.

In a further embodiment of the present methods, R¹ is hydrogen, R² is methyl, R⁴ is hydrogen, and R³, R⁵ and R⁶ are as defined above in Formulas II-V.

In a further embodiment of the present methods, R¹ is hydrogen, R² is methyl, R⁴ is hydrogen, R³ is hydrogen, halogen or methoxy, and the halogen is selected from the group consisting of fluoro or chloro, and R⁵ and R⁶ are as defined above in Formulas II-V.

In a further embodiment of the present methods, R¹ is hydrogen, R² is methyl, R⁴ is hydrogen, R³ is hydrogen, halogen or methoxy, and the halogen is selected from the group consisting of fluoro or chloro, and R⁵ and R⁶ are hydrogen.

In a further embodiment, the present invention relates to a method of treating obesity or an eating disorder, comprising administering to a patient in need thereof a therapeutically effective amount of a compounds of the Formula VI-X, wherein the compound is selected from the group consisting of:

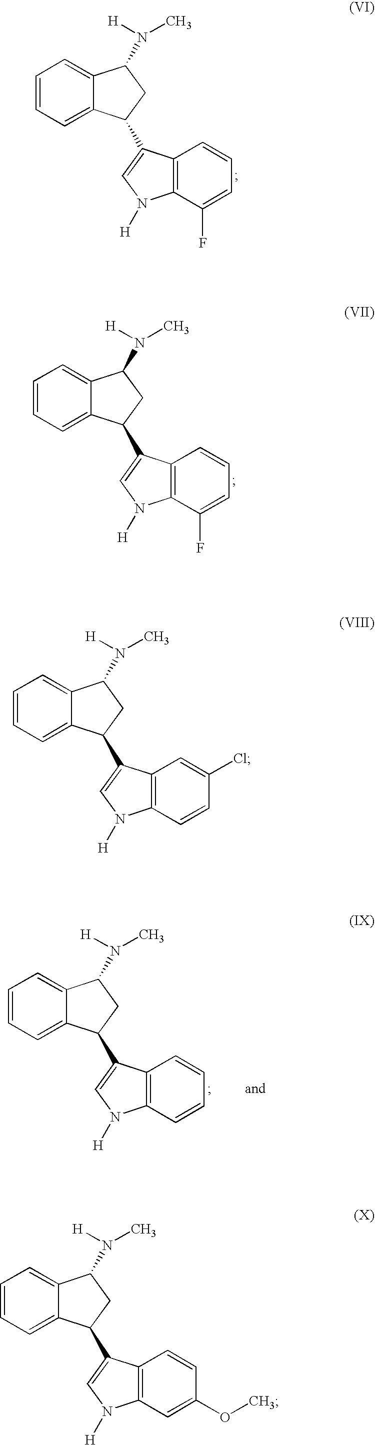

or a pharmaceutically acceptable salt thereof.

As used herein, it is understood that compounds of Formula I-X are meant to include all the examples exemplified herein.

In the present invention, the term Halogen means fluoro, chloro, bromo or iodo.

In the present invention, the term “C₁-C₈ straight or branched alkyl” refers to a saturated hydrocarbon having from one to eight carbon atoms inclusive. Examples of such substituents include, but are not limited to, methyl, ethyl, 1-propyl, 2-propyl, 1-butyl, 2-butyl, 2-methyl-2-propyl, 2-methyl-1-propyl, n-pentyl and n-octyl.

Furthermore, the term “C₃-C₈ cycloalkyl” refers to a saturated cyclohydrocarbon ring having from three to eight carbon atoms inclusive. Included within this term are cyclopropyl, cyclobutyl, cyclopentyl, cyclohexyl, cyclohexyl and cyclooctyl.

The term “C₁-C₅-alkoxy” refers to a saturated alkoxy group having from one to five carbon atoms inclusive with the open valency on the oxygen. Examples of such substituents include, but are not limited to, methoxy, ethoxy, n-butoxy, t-butoxy and n-pentyloxy.

The term “C₁-C₈ straight or branched polyfluoroalkyl” refers to a saturated hydrocarbon having from one to eight carbon atoms inclusive substituted with one or more fluorine atoms. Examples of such substituents include, but are not limited to, trifluoromethyl, pentafluoroethyl, 1-fluoroethyl and 1,2-difluoroethyl and 2,3-difluorooctyl.

A “therapeutically effective amount” of a compound as used herein means an amount sufficient to cure, alleviate or partially arrest the clinical manifestations of a given disease and/or its complications. An amount adequate to accomplish this is defined herein as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspect of the invention. The patient to be treated, i.e. the patient in need thereof, may be a mammal, in particular a human being.

The salts of the invention are may be acid addition salts. The acid addition salts of the invention may be pharmaceutically acceptable salts of the compounds of the invention formed with non-toxic acids. Acid addition salts include salts of inorganic acids as well as organic acids. Examples of suitable inorganic acids include hydrochloric, hydrobromic, hydroiodic, phosphoric, sulfuric, sulfamic, nitric acids and the like. Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977, 66, 2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

Further, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

The compounds of the present invention may have one or more asymmetric centres and it is intended that any isomers (i.e. enantiomers or diastereomers), as separated, pure or partially purified and any mixtures thereof including racemic and diastereomeric mixtures, i.e. a mixture of stereoisomers, are included within the scope of the invention.

Racemic forms can be resolved into the optical antipodes by known methods, for example, by fractional separation of diastereomeric salts thereof with an optically active acid, and liberating the optically active amine compound by treatment with a base. Another method for resolving racemates into the optical antipodes is based upon chromatography on an optically active matrix. The compounds of the present invention may also be resolved by the formation of diastereomeric derivatives. Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981). Optically active compounds can also be prepared from optically active starting materials, by stereoselective synthesis or by enzymatic resolution.

The pharmaceutical compositions of this invention, or those which are manufactured in accordance with this invention, may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used. Tablets may be prepared by mixing the active ingredient with ordinary adjuvants and/or diluents and subsequently compressing the mixture in a conventional tabletting machine. Examples of adjuvants or diluents comprise: corn starch, potato starch, talcum, magnesium stearate, gelatine, lactose, gums, and the like. Any other adjuvants or additives usually used for such purposes such as colourings, flavourings, preservatives, etc., may be used provided that they are compatible with the active ingredients.

Solutions for injections may be prepared by dissolving the active ingredient and possible additives in a part of the solvent for injection, such as sterile water, adjusting the solution to desired volume, sterilizing the solution and filling it in suitable ampules or vials. Any suitable additive conventionally used in the art may be added, such as tonicity agents, preservatives, antioxidants, etc.

Conveniently, the compounds of the invention may be formulated in a unit dosage form, each dosage containing from about 0.01 to about 1000 mg, or from about 0.05 to about 5000, or from about 0.1 to about 1000 mg, the actual dosage may however vary e.g., according to the specific compound. The term “unit dosage form” refers to physically discrete units suitable as unitary dosages for human subjects and other mammals, each unit containing a predetermined quantity of active material calculated to produce the desired therapeutic effect, in association with one or more pharmaceutically acceptable carriers, diluents, excipients or other additives normally used in the art.

The compounds of the invention are effective over a wide dosage range. For example, dosages per day normally fall within the range of about 0.01 to about 100 mg/kg of body weight, or within the range of about 0.1 to about 75 mg/kg. However, it will be understood that the amount of the compound actually administered will be determined by a physician, according to the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

### Experimental Section

C-MS Methods A and B, general: Solvent system: A=water/TFA (100:0.05) and B=water/acetonitrile/TFA (5:95:0.035) (TFA=trifluoroacetic acid). Purity was determined by integration of the UV (254 nm) and ELSD trace and retention times (RT) are expressed in minutes. MS instruments are from PESciex (API), equipped with APPI-source and operated in positive ion mode.

Method A: API 150EX and Shimadzu LC8/SLC-10A LC system. Column: 30×4.6 mm Waters Symmetry C18 with 3.5 μM particles operated at room temperature. Linear Gradient elution with 90% A to 100% B in 4 min and a flow rate of 2 ml/min.

Method B: API 150EX and Shimadzu LC8/SLC-10A LC system. Column: 30×4.6 mm Waters Symmetry C18 with 3.5 μM particles operated at 40° C. Linear Gradient elution with 90% A to 100% B in 2.4 min and a flow rate of 3.3 ml/min.

LC-MS TOF (TOF=time of flight) Method C: micromass LCT 4-ways MUX equipped with a Waters 2488/Sedex 754 detector system. Column: 30×4.6 mm Waters Symmetry C18 column with 3.5 μm particle size operated at room temperature. Linear Gradient elution with 90% A to 100% B in 4 min and a flow rate of 2 ml/min. Purity was determined by integration of the UV (254 nm) and ELSD trace and retention times (RT) are expressed in minutes.

¹H NMR and ¹³C NMR spectra were recorded on a Bruker DRX 500 at 500.13 MHz and 125.67, respectively. Deuterated chloroform (99.8% D) or dimethylsulfoxide (99.9% D) were used as solvents. TMS was used as internal reference standard. Chemical shifts are expressed as ppm values. The following abbreviations are used for multiplicity of NMR signals: s=singlet, d=doublet, t=triplet, q=quartet, qv=quintet, h=heptet, dd=double doublet, dt=double triplet, dq=double quartet, tt=triplet of triplets, m=multiplet, b=broad.

### EXAMPLES

### Synthesis of 3-bromo-indan-1-one

390 g (2.2 mol) N-bromosuccinimde (powder with no lumps) and 0.5 g benzoyl peroxide were added to 264 g indan-1-one in 1500 mL CCl₄ and refluxed with mechanical stirring for 1.5 hours. The colour of the reaction mixture suddenly changed to yellow and all N-bromosuccinimde (heavier than CCl₄) was converted to succinimide (lighter than CCl₄). The reaction mixture was cooled to 20° C., filtered and concentrated in vacuo. Crude 3-bromo-indan-1-one was dissolved in 600 mL ethyl acetate/heptane (1:2), cooled 2 hours on an ice bath and then left in a freezer over night to give 257 g crystals of 3-bromo-indan-1-one (62% yield).

### Synthesis of 3-(7-fluoro-1H-indol-3-yl)-indan-1-one (Method 1)

Triethylamine (4.5 mL; 32 mmol; 1.2 equiv.) was added to 3-bromo-indan-1-one (5.6 g; 27 mmol) in 100 mL THF at 0° C. and stirred 1 h at r.t. The reaction mixture was filtered to remove triethylammonium bromide and concentrated in vacuo to give inden-1-one.

7-Fluoroindole (3.3 g, 22 mmol) and Sc(OTf)₃ (550 mg, 5 mol %) were added to inden-1-one in 100 mL CH₂Cl₂ at 0° C. The reaction mixture was allowed to warm to room temperature over night. 100 mL ethyl acetate was added and the mixture was filtered through a silica gel plug and concentrated in vacuo. After flash chromatography (heptane/ethyl acetate, silica gel) 5.9 g 3-(7-fluoro-1H-indol-3-yl)-indan-1-one (82%) was isolated.

The following compounds were synthesized in a similar way:

3-(1H-indol-3-yl)-indan-1-one;

3-(1-Methyl-1H-indol-3-yl)-indan-1-one;

3-(5-Fluoro-1H-indol-3-yl)-indan-1-one;

3-(6-Fluoro-1H-indol-3-yl)-indan-1-one;

3-(6-Methoxy-1H-indol-3-yl)-indan-1-one;

3-(5-Fluoro-2-methyl-1H-indol-3-yl)-indan-1-one;

3-(4-Chloro-1H-indol-3-yl)-indan-1-one;

3-(5-Chloro-1H-indol-3-yl)-indan-1-one;

3-(7-Chloro-1H-indol-3-yl)-indan-1-one;

5-Fluoro-3-(7-methyl-1H-indol-3-yl)-indan-1-one;

3-(6-Bromo-1H-indol-3-yl)-5-fluoro-indan-1-one;

3-(4-Chloro-1H-indol-3-yl)-6-fluoro-indan-1-one;

3-(4,6-Difluoro-1H-indol-3-yl)-6-fluoro-indan-1-one;

3-(4-Chloro-1H-indol-3-yl)-6-methoxy-indan-1-one;

3-(5,6-Difluoro-1H-indol-3-yl)-6-methoxy-indan-1-one

5-Chloro-3-(5-fluoro-1H-indol-3-yl)-indan-1-one;

3-(6-Chloro-3-oxo-indan-1-yl)-1H-indole-5-carbonitrile;

6-Chloro-3-(5-methoxy-1H-indol-3-yl)-indan-1-one; and

6-Chloro-3-(7-methoxy-1H-indol-3-yl)-indan-1-one.

### Synthesis of cis-3-(1H-indol-3-yl)-indan-1-ol (Method 2)

NaBH₄ (6.2 g, 163 mmol, 2 equiv.) was added to 3-(1H-indol-3-yl)-indan-1-one (20 g, 80.9 mmol) in 200 mL methanol and 100 mL THF at 0° C. The reaction mixture was allowed to warm to room temperature over night. Aqueous work up gave racemic cis-3-indolyl-indan-1-ol (quantitative).

The following compounds were synthesized in a similar way:

cis-3-(6-Fluoro-1H-indol-3-yl)-indan-1-ol;

cis-3-(6-Methoxy-1H-indol-3-yl)-indan-1-ol;

cis-3-(5-Chloro-1H-indol-3-yl)-indan-1-ol;

cis-3-(4-Chloro-1H-indol-3-yl)-6-fluoro-indan-1-ol; and

cis-6-Chloro-3-(5-methoxy-1H-indol-3-yl)-indan-1-ol.

### Synthesis of butyric acid (1R,3S)-3-(1H-indol-3-yl)-indan-1-yl ester and (1S,3R)-3-(1H-indol-3-yl)-indan-1-ol (Method 3)

Novozym 435 (1 g) (Available from Novozymes A/S, Krogshoejvej 36, 2880 Bagsvaerd, Denmark) and vinyl butyrate (20.5 mL, 162 mmol) were added to racemic cis-3-indolyl-indan-1-ol (80.9 mmol) in 200 mL toluene. The reaction mixture was shaken for 2 days under argon until ¹H-NMR shows 50% conversion. The reaction mixture was filtered and concentrated in vacuo. After flash chromatography (heptane/ethyl acetate, silica gel) 13.49 g butyric acid (1R,3S)-3-(1H-indol-3-yl)-indan-1-yl ester and 9.78 g (1S,3R)-3-(1H-indol-3-yl)-indan-1-ol were isolated.

The following compounds were synthesized in a similar way:

Butyric acid (1R,3S)-3-(7-fluoro-1H-indol-3-yl)-indan-1-yl ester;

(1S,3R)-3-(7-Fluoro-1H-indol-3-yl)-indan-1-ol;

(1S,3R)-3-(5-Chloro-1H-indol-3-yl)-indan-1-ol; and

(1S,3R)-3-(6-Methoxy-1H-indol-3-yl)-indan-1-ol.

### Synthesis of (1R,3S)-3-(1H-indol-3-yl)-indan-1-ol (Method 4)

3 mL 30% NaOMe in methanol was added to 13.49 g butyric acid (1R,3S)-3-(1H-indol-3-yl)-indan-1-yl ester in 100 mL methanol. TLC showed full conversion after 1.5 hours. 1.5 g solid NH₄Cl and 50 mL water were added. Methanol was removed in vacuo and after aqueous work up, (1R,3S)-3-(1H-indol-3-yl)-indan-1-ol (37.9 mmol) was obtained.

The following compound was synthesized in a similar way:

(1R,3S)-3-(7-Fluoro-1H-indol-3-yl)-indan-1-ol.

### Synthesis of (R)-3-(1H-indol-3-yl)-indan-1-one (Method 5)

Dess-Martin Periodane (1.08 g, 2.55 mmol) in 10 mL CH₂Cl₂ was added to (1S,3R)-3-(1H-Indol-3-yl)-indan-1-ol (2.52 mmol) in 10 mL CH₂Cl₂ at 0° C. The reaction mixture was allowed to warm to room temperature and stirred 40 min—TLC showed full conversion. To the reaction mixture ethyl acetate and sat. NaHCO₃ was added. The organic phase was isolated, washed with 2N NaOH and brine, dried over MgSO₄ and concentrated in vacuo. A quantitative yield of (R)-3-(1H-indol-3-yl)-indan-1-one was obtained after flash chromatography (heptane/ethyl acetate, silica gel).

The following compounds were synthesized in a similar way:

(R)-3-(7-Fluoro-1H-indol-3-yl)-indan-1-one; and

(S)-3-(7-Fluoro-1H-indol-3-yl)-indan-1-one.

### Synthesis of 3-((1S,3S)-3-azido-indan-1-yl)-1H-indole (Method 6)

1,8-Diazabicyclo[5.4.0]undec-7-ene (4.8 mL, 32.1 mmol, 1.45 equiv) was added to (1R,3S)-3-(1H-indol-3-yl)-indan-1-ol (22.2 mmol) and diphenyl phosphoryl azide (6.0 mL, 27.8 mmol, 1.25 equiv) in 150 mL dry THF at 0° C. The reaction was stirred 0.5 hours at 0° C., then 2 hours at room temperature—TLC showed full conversion. The reaction mixture was poured into water and extracted with ethyl acetate. The organic phase was washed with 100 mL 0.5 N HCl, sat. NaHCO₃, dried over MgSO₄ and concentrated in vacuo to give the 3-((1S,3S)-3-azido-indan-1-yl)-1H-indole.

The following compound was synthesized in a similar way:

3-((1R,3R)-3-Azido-indan-1-yl)-6-methoxy-1H-indole.

### Synthesis of [3-((1R,3R)-3-azido-indan-1-yl)-5-chloro-indol-1-yl]-phosphonic acid diphenyl ester (Method 7)

Diphenyl phosphoryl azide (23 g, 85 mmol) was added to (1S,3R)-3-(5-chloro-1H-indol-3-yl)-indan-1-ol (10 g, 35 mmol) in 150 mL dry THF at 5° C. 1,8-diazabicyclo[5.4.0]undec-7-ene (13.7 mL, 92 mmol) was added over 0.5 hours. The reaction mixture was stirred over night while warming to room temperature. The reaction mixture was poured into brine and extracted with Et₂O. The organic phase was washed with 100 mL 0.1 N HCl, 0.1 N NaOH, dried over MgSO₄ and concentrated in vacuo. After flash chromatography (heptane/ethyl acetate, silica gel), 19.1 g [3-((1R,3R)-3-azido-indan-1-yl)-5-chloro-indol-1-yl]-phosphonic acid diphenyl ester was isolated.

The following racemic compounds were synthesized in a similar way:
[3-(3-Azido-indan-1-yl)-indol-1-yl]-phosphonic acid diphenyl ester;
[3-(3-Azido-indan-1-yl)-6-fluoro-indol-1-yl]-phosphonic acid diphenyl ester;
[3-(3-Azido-indan-1-yl)-6-methoxy-indol-1-yl]-phosphonic acid diphenyl ester;
[3-(3-Azido-5-fluoro-indan-1-yl)-4-chloro-indol-1-yl]-phosphonic acid diphenyl ester; and
[3-(3-Azido-5-chloro-indan-1-yl)-5-methoxy-indol-1-yl]-phosphonic acid diphenyl ester.

### Synthesis of 3-((1R,3R)-3-azido-indan-1-yl)-6-methoxy-1-(toluene-4-sulfonyl)-1H-indole (Method 8)

1 g Sodium hydride (60% in mineral oil) was added to 2 g 3-((1R,3R)-3-azido-indan-1-yl)-6-methoxy-1H-indole in 50 mL dry THF at 5° C. and the reaction mixture was stirred 1 hour at 5° C. 2 g p-toluene sulfonic acid chloride was added in portions at 5° C. and stirring was continued for another 4 hours. Ice was added and after 1 hour water and ethyl acetate was added. The organic phase was separated and washed with brine, dried over MgSO₄ and concentrated in vacuo. After flash chromatography (heptane/ethyl acetate, silica gel), 2.5 g 3-((1R,3R)-3-azido-indan-1-yl)-6-methoxy-1-(toluene-4-sulfonyl)-1H-indole was isolated.

The following compound was synthesized in a similar way:

3-((1R,3R)-3-Azido-indan-1-yl)-1-(toluene-4-sulfonyl)-1H-indole.

### Synthesis of (1R,3R)-3-(5-chloro-1H-indol-3-yl)-indan-1-ylamine (Method 9)

32 g Trimethyl phosphine was added over 2 hours to 19 g [3-((1R,3R)-3-azido-indan-1-yl)-5-chloro-indol-1-yl]-phosphonic acid diphenyl ester in 100 mL pyridine and 36 mL 9N ammonium hydroxide at room temperature. The reaction mixture was stirred over night at room temperature and concentrated in vacuo. Ethyl acetate and water were added. The mixture was made basic with aqueous NaOH and filtered. The organic phase was concentrated in vacuo and was dissolved in ethyl acetate again. The ethyl acetate solution was extracted with 2×250 mL 2N methanesulfonic acid. The aqueous phase was made basic with 9N NaOH to form a precipitate, which was subjected to flash chromatography (ethyl acetate, methanol, triethylamine, silica gel) to give 0.6 g (1R,3R)-3-(5-chloro-1H-indol-3-yl)-indan-1-ylamine. The ethyl acetate phase from above was concentrated in vacuo and was dissolved in 100 mL methanol and 10 mL 30% NaOMe in methanol was added. The reaction mixture was stirred 2 hours at room temperature, concentrated in vacuo and purified by flash chromatography (ethyl acetate, methanol, triethylamine, silica gel) to give further 0.45 g (1R,3R)-3-(5-chloro-1H-indol-3-yl)-indan-1-ylamine.

### Synthesis of Example 7

### [(1S,3R)-3-(1H-indol-3-yl)-indan-1-yl]-methyl-amine (Method 10)

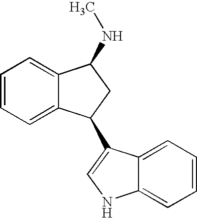

(R)-3-(1H-indol-3-yl)-indan-1-one, methyl amine (5 mL 2M in THF) and 1 mL tetraethoxy silane in 10 mL methanol were stirred 11 minutes at 150° C. under microwave irradiation using the Emry Optimizer™ instrument. PtO₂ (10 mg) was added and the reaction mixture was stirred under 1 atm. hydrogen over night at room temperature, filtered and concentrated in vacuo. [(1S,3R)-3-(1H-Indol-3-yl)-indan-1-yl]-methyl-amine was isolated in 66% yield after flash chromatography (ethyl acetate, methanol, triethyl amine, silica gel).

The following compounds were synthesized in a similar way:

### Example 1

### Racemic cis-[3-(5-Fluoro-2-methyl-1H-indol-3-yl)-indan-1-yl]-methyl-amine

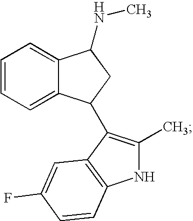

### Example 2

### Racemic cis-[3-(7-Methoxy-1H-indol-3-yl)-indan-1-yl]-methyl-amine

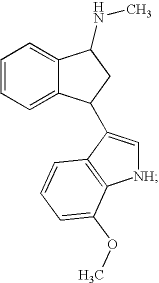

### Example 3

### Racemic cis-Methyl-[3-(1-methyl-1H-indol-3-yl)-indan-1-yl]-amine

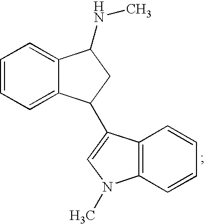

### Example 4

### Racemic cis-[3-(5-Fluoro-1H-indol-3-yl)-indan-1-yl]-methyl-amine

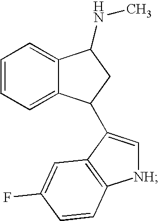

### Example 5

### Racemic cis-[3-(7-Chloro-1H-indol-3-yl)-indan-1-yl]-methyl-amine

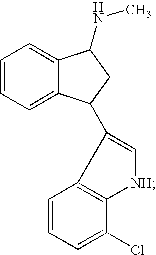

### Example 6

### Racemic cis-[3-(4-Chloro-1H-indol-3-yl)-indan-1-yl]-methyl-amine

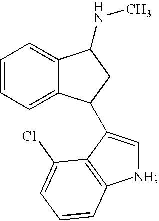

### Example 8

### [(1S,3R)-3-(7-Fluoro-1H-indol-3-yl)-indan-1-yl]-methyl-amine

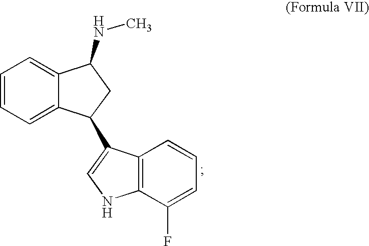

Preparative scale chiral SFC purification (Method 15): Method: Column: Chiralcel OJ-H (2×25 cm) operated at room temperature. Chromatography was carried out using 35% of methanol with 0.1% (v/v) diethylamine as modifier in CO2 (100 bar) and a flow rate 50 ml/min.

Analytical Chiral SFC: Method: Column. 250×4.6 mm Chiralcel AD-H with 5 μM particles operated at room temperature. Chromatography was carried out using 40% of ethanol with 0.1% (v/v) diethylamine as modifier in CO2 (100 bar) and a flow rate 3 ml/min. Detection at 220 nm. RTₘₐⱼₒᵣ=2.52 min, RTₘᵢₙₒᵣ=3.29 min, >99.5% ee.

LC/MS: Method A: RT=1.85 min. UV-purity=99.30%, ELSD purity=96.78%

¹H-NMR (hydrobromide salt; d₆-DMSO): δ 2.23-2.27 (m, 1H), 2.71 (s, 3H), 2.93-2.95 (m, 1H), 4.62 (dd, J=7.8 Hz, 10.2 Hz, 1H), 4.88 (t, J=8.3 Hz, 1H), 6.87-6.96 (m, 3H), 7.12 (d, J=7.7 Hz, 1H), 7.30 (t, J=14.8 Hz, 1H), 7.35-7.38 (m, 2H), 7.72 (d, J=7.6 Hz, 1H), 11.5 (bs, 1H).

### Example 9

### [(1R,3S)-3-(7-Fluoro-1H-indol-3-yl)-indan-1-yl]-methyl-amine

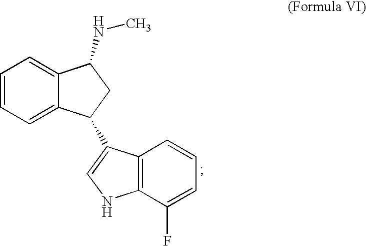

Preparative scale chiral SFC purification (Method 15): Method: Column: Chiralcel OJ-H (2×25 cm) operated at room temperature. Chromatography was carried out using 35% of methanol with 0.1% (v/v) diethylamine as modifier in CO2 (100 bar) and a flow rate 50 ml/min.

Analytical Chiral SFC: Method: Column: 250×4.6 mm Chiralcel AD-H with 5 μM particles operated at room temperature. Chromatography was carried out using 40% of ethanol with 0.1% (v/v) diethylamine as modifier in CO2 (100 bar) and a flow rate 3 ml/min. Detection at 220 nm. RTₘₐⱼₒᵣ=3.36 min, RTₘᵢₙₒᵣ=2.46 min, >99.5% ee.

LC/MS: Method B: RT=0.90 min. UV-purity=99.80%, ELSD purity=96.08%

¹H-NMR (d₆-DMSO): δ 1.89-1.96 (m, 1H), 2.15 (bs, 1H), 2.40 (s, 3H), 2.75-2.80 (m, 1H), 4.17 (dd, J=7.3 Hz, 8.7 Hz, 1H), 4.44 (dd, J=7.5 Hz, 10.4 Hz, 1H), 6.82-6.90 (m, 3H), 7.07-7.11 (m, 2H), 7.20 (t, J=7.4 Hz, 1H), 7.26 (d, J=2.2 Hz, 1H), 7.42 (d, J=7.5 Hz, 1H), 11.4 (bs, 1H).

¹³C-NMR (d₆-DMSO): δ 33.7, 39.4, 63.3, 106.1 (J_{CF}=16 Hz), 115.7, 118.5, 118.8, 124.0, 124.1, 124.3, 124.8, 124.9, 126.6, 127.2, 130.8, 145.7, 146.2, 149.7 (J_{CF}=243 Hz).

### Example 11

### Racemic cis-[6-Chloro-3-(7-methoxy-1H-indol-3-yl)-indan-1-yl]-methyl-amine

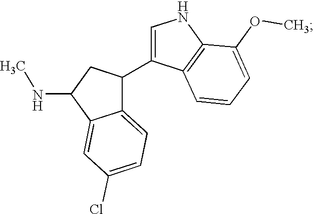

### Example 12

### Racemic cis-[3-(4-Chloro-1H-indol-3-yl)-6-methoxy-indan-1-yl]-methyl-amine

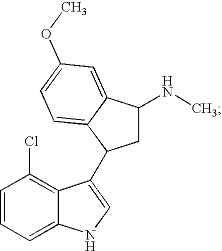

### Example 13

### Racemic cis-[3-(5,6-Difluoro-1H-indol-3-yl)-6-methoxy-indan-1-yl]-methyl-amine

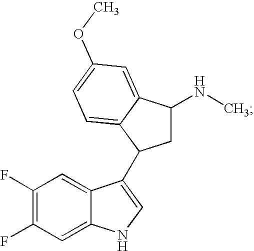

### Example 14

### Racemic cis-3-(6-Chloro-3-methylamino-indan-1-yl)-1H-indole-5-carbonitrile

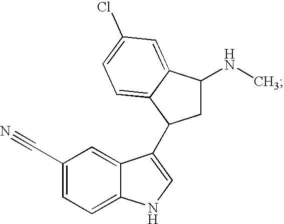

### Example 15

### Racemic cis-[5-Fluoro-3-(7-methyl-1H-indol-3-yl)-indan-1-yl]-methyl-amine

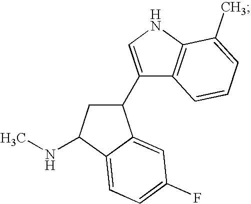

### Example 16

### Racemic cis-[3-(6-Bromo-1H-indol-3-yl)-5-fluoro-indan-1-yl]-methyl-amine

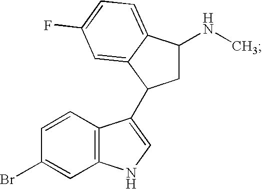

### Example 23

### Racemic cis-[3-(4,6-Difluoro-1H-indol-3-yl)-6-fluoro-indan-1-yl]-methyl-amine

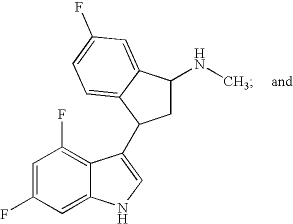

### Example 24

### Racemic cis-[5-Chloro-3-(5-fluoro-1H-indol-3-yl)-indan-1-yl]-methyl-amine

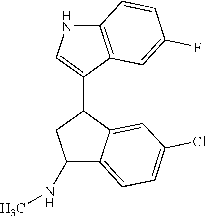

### Synthesis of Example 19

### cis-3-(3-Piperidin-1-yl-indan-1-yl)-1H-indole (Method 11)

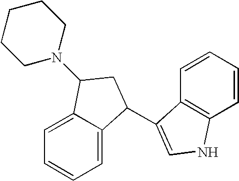

Sodium cyanoborohydride (61 mg; 0.97 mmol) was added to 3-(1H-indol-3-yl)-indan-1-one (200 mg; 0.81 mmol) and piperidine (344 mg; 4.05 mmol) in 3 mL methanol and 0.5 mL acetic acid. The reaction mixture was stirred 30 minutes at 150° C. under microwave irradiation using the Emry Optimizer™ instrument. The reaction mixture was poured into water and made basic with 27% aqueous NaOH. The mixture was extracted with ethyl acetate. The organic phase was dried over MgSO₄ and concentrated in vacuo. cis-3-(3-Piperidin-1-yl-indan-1-yl)-1H-indole was obtained after flash chromatography (ethyl acetate, heptane, triethylamine, silica gel).

The following compounds were synthesized in a similar way:

### Example 20

### Racemic cis-3-(3-Pyrrolidin-1-yl-indan-1-yl)-1H-indole

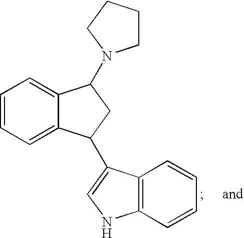

### Example 21

### Racemic cis-3-(3-Morpholin-4-yl-indan-1-yl)-1H-indole

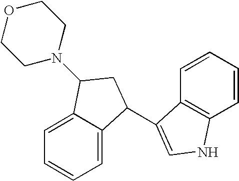

### Synthesis of Example 10

### [(1R,3R)-3-(1H-indol-3-yl)-indan-1-yl]-methyl-amine (Method 13)

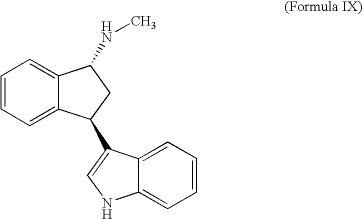

Dimethylbromoborane (1.77 mL, 1.05 equiv) (Synthesized according to Nöth, H., Vahrenkamp, H. Journal of Organometallic Chemistry 11(1968), 399-405) was added to 3-((1R,3R)-3-azido-indan-1-yl)-1-(toluene-4-sulfonyl)-1H-indole (17.3 mmol) in 100 mL 1,2-dichloro-ethane under argon at 0° C. The reaction mixture was warmed to room temperature and stirred 2.5 hours. 1 mL Ethanol was added. The reaction mixture was extracted with ethyl acetate and 0.5N aqueous NaOH. The organic phase was washed with brine, dried over MgSO₄, concentrated in vacuo to give methyl-{(1R,3R)-3-[1-(toluene-4-sulfonyl)-1H-indol-3-yl]-indan-1-yl}-amine after flash chromatography (ethyl acetate, methanol, triethylamine, silica gel). Methyl-{(1R,3R)-3-[1-(toluene-4-sulfonyl)-1H-indol-3-yl]-indan-1-yl}-amine was dissolved in 8 mL acetone and 20 mL methanol. 8 mL 28% Aqueous NaOH was added and the reaction mixture was stirred in two portions at 120° C. for 10 minutes under microwave irradiation using the Emry Optimizer™ instrument. The reaction mixture was poured into 250 mL water and a precipitate was formed. Recrystallization gave 2.15 g [(1R,3R)-3-(1H-indol-3-yl)-indan-1-yl]-methyl-amine.

Chiral SFC: Method: Column: 250×4.6 mm Chiralcel OJ-H with 5 μM particles operated at room temperature. Chromatography was carried out using 30% of 0.1% (v/v) diethylamin in ethanol as modifier, a pressure of 20 MPa and a flow rate 3 ml/min. Detection at 230 nm. RTₘₐⱼₒᵣ=2.40 min, RTₘᵢₙₒᵣ=2.93 min, 95.9% ee.

LC/MS: Method A: RT=1.63 min. UV-purity=98.28%, ELSD purity=99.61%

¹H-NMR (d₆-DMSO): δ 2.35 (m, 5H), 4.17 (dd, J=3.8 Hz, 6.6 Hz, 1H), 4.75 (t, J=7.5 Hz, 1H), 6.90 (t, J=7.1 Hz, 1H), 7.00 (m, 3H), 7.14 (t, J=7.3 Hz, 1H), 7.19 (t, J=7.3 Hz, 1H), 7.26 (d, J=8.0 Hz, 1H) 7.34 (d, J=7.3 Hz, 1H), 7.38 (d, J=7.3 Hz, 1H), 10.80 (bs, 1H).

¹³C-NMR (d₆-DMSO): δ 34.2, 39.9, 41.3, 63.6, 111.9, 118.0, 118.5, 119.1, 121.3, 122.4, 124.8, 125.0, 126.4, 126.7, 127.6, 137.1, 145.3, 147.2.

The following compound was synthesized in a similar way:

### Example 27

### [(1R,3R)-3-(6-Methoxy-1H-indol-3-yl)-indan-1-yl]-methyl-amine

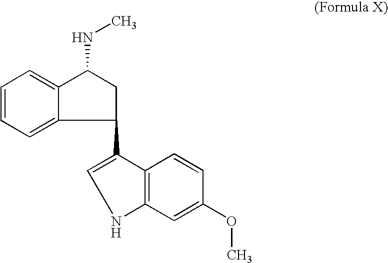

LC/MS: Method A: RT=1.60 min. UV-purity=98.52%, ELSD purity=99.53%

¹H-NMR (CDCl₃): δ 2.41 (ddd, J=3.8 Hz, 7.5 Hz, 12.8 Hz, 1H), 2.50 (t, J=6.8 Hz, 1H), 2.53 (s, 3H), 3.83 (s, 3H), 4.27 (dd, J=3.8 Hz, 6.8 Hz, 1H), 4.79 (t, J=7.5 Hz, 1H), 6.72 (m, 2H), 6.85 (s, 1H), 7.2 (m, 4H), 7.40 (d, J=7.3 Hz, 1H), 7.82 (bs, 1H).

### Synthesis of Example 25

### Racemic trans-[6-Chloro-3-(5-methoxy-1H-indol-3-yl)-indan-1-yl]-methyl-amine (Method 13)

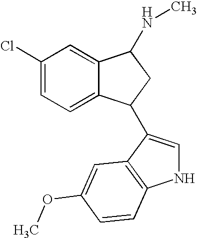

0.15 mmol Dimethylbromoborane (Synthesized according to Nöth, H., Vahrenkamp, H. Journal of Organometallic Chemistry 11(1968), 399-405) in 0.5 mL dry 1,2-dichloroethane was added to approximately 0.1 mmol [3-(3-Azido-6-chloro-indan-1-yl)-indol-1-yl]-phosphonic acid diphenyl ester in 2 mL dry 1,2-dichloroethane. The reaction mixture was stirred 3 hours at room temperature. The reaction was quenched by the addition of 1 mL 1 N NaOH and 1 mL brine. The mixture was extracted with ethyl acetate. The organic phase was concentrated in vacuo. The residue was treated with 3 mL 1M sodium methoxide in methanol for 3 h at room temperature. 1 mL acetic acid was added and [6-Chloro-3-(1H-indol-3-yl)-indan-1-yl]-methyl-amine was isolated after preparative HPLC.

The following compounds were synthesized in a similar way:

### Example 17

### Racemic trans-[3-(6-Fluoro-1H-indol-3-yl)-indan-1-yl]-methyl-amine

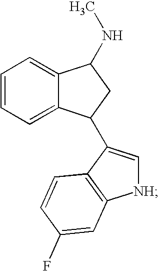

### Example 18

### Racemic trans-[3-(6-Methoxy-1H-indol-3-yl)-indan-1-yl]-methyl-amine

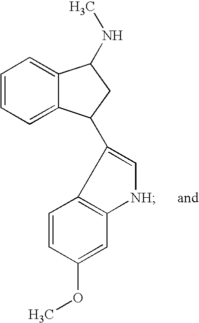

### Example 22

### Racemic trans-[3-(4-Chloro-1H-indol-3-yl)-6-fluoro-indan-1-yl]-methyl-amine

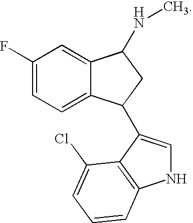

### Synthesis of Example 26

### [(1R,3R)-3-(5-chloro-1H-indol-3-yl)-indan-1-yl]-methyl-amine (Method 14)

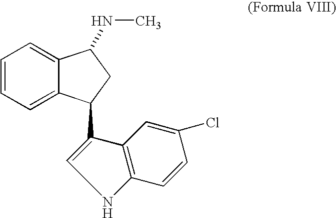

0.73 g (1R,3R)-3-(5-chloro-1H-indol-3-yl)-indan-1-ylamine was suspended in 200 mL 1,2-dichloro-ethane, 100 mL water and 0.5 mL 9N NaOH. 0.25 mL Methyl chloroformate (1.2 equiv) and 50 mg Bu₄NBr were added. The reaction mixture was stirred 30 minutes at room temperature. The organic phase was dried over MgSO₄ and concentrated in vacuo to give 0.6 g [(1R,3R)-3-(5-chloro-1H-indol-3-yl)-indan-1-yl]-carbamic acid methyl ester. 0.6 g [(1R,3R)-3-(5-Chloro-1H-indol-3-yl)-indan-1-yl]-carbamic acid methyl ester was obtained after aqueous work up. [(1R,3R)-3-(5-Chloro-1H-indol-3-yl)-indan-1-yl]-carbamic acid methyl ester was dissolved in 250 mL dry THF and 0.6 g LiAlH₄ was added. The reaction mixture was refluxed for 2 hours. The reaction was quenched with 2 mL water, filtered and concentrated in vacuo to give an oil. The oil was dissolved in ethyl acetate and 350 mg [(1R,3R)-3-(5-chloro-1H-indol-3-yl)-indan-1-yl]-methyl-amine precipitated on standing at room temperature over night.

Chiral SFC: Method: Column: 250×4.6 mm Chiralcel AD-H with 5 μM particles operated at room temperature. Chromatography was carried out using 30% of 0.1% (v/v) diethylamin in ethanol as modifier, a pressure of 20 MPa and a flow rate 3 ml/min. Detection at 230 nm. RTₘₐⱼₒᵣ=2.89 min, RTₘᵢₙₒᵣ=3.84 min, 88.6% ee.

LC/MS: Method C: RT=1.54 min. UV-purity=95.21%, ELSD purity=100%

¹³H-NMR (CDCl₃): δ 2.38-2.52 (m, 2H), 2.53 (s, 3H), 4.27 (dd, J=3.9 Hz, 6.6 Hz), 4.77 (t, J=7.4), 6.82 (s, 1H), 7.12 (dd, J=2.0 hZ, 8.5 Hz, 1H), 7.20-7.27 (m, 3H) 7.37 (d, J=2.0 Hz), 7.42 (d, J=7.4 Hz), 8.16 (bs, 1H).

### Chiral separation of racemic cis-3-(7-Fluoro-1H-indol-3-yl)-indan-1-yl-methyl-amine (Method 15)

20 g of racemic cis-3-(7-Fluoro-1H-indol-3-yl)-indan-1-yl-methyl-amine (from Method 10) was purified by chiral SFC to give 9.3 g of [(1S,3R)-3-(7-Fluoro-1H-indol-3-yl)-indan-1-yl]-methyl-amine with >99.5% ee and 9.5 g of [(1R,3S)-3-(7-Fluoro-1H-indol-3-yl)-indan-1-yl]-methyl-amine with >99.5% ee.

Preparative scale chiral SFC purification method: Column: Chiralcel OJ-H (2×25 cm) with 5 μM particles operated at 35° C. Chromatography was carried out using 35% of methanol with 0.1% (v/v) diethylamine as modifier in CO2 (100 bar) and a flow rate 50 ml/min.

### Fractional crystallization of racemic cis-3-(7-Fluoro-1H-indol-3-yl)-indan-1-yl-methyl-amine with an optically active acid (Method 16)

To a solution of the racemate cis-3-(7-Fluoro-1H-indol-3-yl)-indan-1-yl-methyl-amine (409 mg/2 ml EtOH) was added a solution of Di-p-toluoyl-D-tartaric acid (1 eq; 564 mg/2 ml Acetone). The mixture was heated to 50° C. and stirred for 15 min. Evaporated the solvent and triturated with acetone (2 ml). The white solid (84% ee) was collected and stirred with 10 ml hot EtOH for 30 min. The remaining white solid was collected and converted to free base to give [(1S,3R)-3-(7-Fluoro-1H-indol-3-yl)-indan-1-yl]-methyl-amine (93% ee).

TABLE A
Measured molecular mass (M + H⁺), measured
HPLC-retention time (Rₜ, min) and UV- and ELSD-purities (%).

| Example | | Rₜ | UV | ELSD | |
|---|---|---|---|---|---|
| No. | M + H⁺ | (min.) | purity % | purity % | Method |
| 1 | 295.2 | 1.82 | 98.6 | 99.9 | A |
| 2 | 293.2 | 1.79 | 96.7 | 99.2 | A |
| 3 | 277.2 | 1.91 | 92.5 | 99.9 | A |
| 4 | 281.1 | 1.78 | 91.5 | 99.6 | A |
| 5 | 297.1 | 1.94 | 89.7 | 99.6 | A |
| 6 | 297.1 | 1.98 | 89.3 | 99.6 | A |
| 7 | 263.1 | 1.66 | 91.1 | 99.6 | A |
| 8 | 281.1 | 1.85 | 99.3 | 96.8 | A |
| 9 | 281.0 | 1.89 | 95.2 | 99.5 | A |
| 10 | 263.1 | 1.64 | 91.9 | 100.0 | A |
| 11 | 327.1 | 1.97 | 92.9 | 99.9 | A |
| 12 | 327.1 | 2.02 | 74.5 | 99.3 | A |
| 13 | 329.1 | 1.94 | 88.0 | 99.7 | A |
| 14 | 322.1 | 1.83 | 89.2 | 99.9 | A |
| 15 | 295.2 | 1.93 | 92.2 | 99.1 | A |
| 16 | 359.0 | 2.07 | 87.7 | 99.4 | A |
| 17 | 281.1 | 1.77 | 96.8 | 99.6 | A |
| 18 | 293.2 | 1.75 | 96.2 | 100.0 | A |
| 19 | 317.1 | 1.85 | 85.1 | 99.0 | A |
| 20 | 303.1 | 1.82 | 98.3 | 98.9 | A |
| 21 | 319.0 | 1.72 | 94.3 | 99.4 | A |
| 22 | 315.1 | 1.88 | 90.2 | 99.8 | A |
| 23 | 317.1 | 1.89 | 87.8 | 99.3 | A |
| 24 | 315.1 | 1.90 | 89.3 | 98.6 | A |
| 25 | 327.1 | 1.89 | 82.7 | 99.3 | A |
| 26 | 297.2 | 0.94 | 94.5 | 99.4 | B |
| 27 | 293.1 | 1.60 | 98.5 | 99.5 | A |

### Transporter Inhibition Assays

### Measurements of [³H]-5-HT Uptake Into Rat Cortical Synaptosomes

Whole brains from male Wistar rats (125-225 g), excluding cerebellum, are homogenized in 0.40 M sucrose supplemented with 1 mM nialamid with a glass/teflon homogenizer. The homogenate is centrifuged at 1000×g for 10 rain at 4° C. The pellet is discarded and the supernatant is centrifuged at 40.000×g for 20 min. The final pellet is homogenized in assay buffer (0.5 mg original tissue/well). Test compounds (or buffer) and 10 nM [³H]-5-HT are added to 96 well plates. Composition of assay buffer: 123 mM NaCl, 4.82 mM KCl, 0.973 mM CaCl₂, 1.12 mM MgSO₄, 12.66 mM Na₂HPO₄, 2.97 mM NaH₂PO₄, 0.162 mM EDTA, 2 g/l glucose and 0.2 g/l ascorbic acid. Buffer is oxygenated with 95% 0₂/5% C0₂ for 10 min. The incubation is started by adding tissue to a final assay volume of 0.2 mL. After 15 min incubation with radioligand at 37° C., samples are filtered directly on Unifilter GF/C glass fiber filters (soaked for 30 min in 0.1% polyethylenimine) under vacuum and immediately washed with 1×0.2 ml assay buffer. Non-specific uptake is determined using citalopram (10 μM final concentration). Citalopram is included as reference in all experiments as dose-response curve.

### Measurements of [³H]Noradrenaline Uptake Into Rat Cortical Synaptosomes

Fresh occipital-, temporal-og parietal cortex from male Wistar rats (125-225 g) are homogenized in 0.4M sucrose with a glass/teflon homogenizer. The homogenate is centrifuged at 1000×g for 10 min at 4° C. The pellet is discarded and the supernatant is centrifuged at 40.000×g for 20 min. The final pellet is homogenized in this assay buffer: 123 mM NaCl, 4.82 mM KCl, 0.973 mM CaCl₂, 1.12 mM MgSO₄, 12.66 mM Na₂HPO₄, 2.97 mM NaH₂PO₄, 0.162 mM EDTA, 2 g/l glucose and 0.2 g/l ascorbic acid (7.2 mg original tissue/mL=1 mg/140 μl). Buffer is oxygenated with 95% 0₂/5% C0₂ for 10 min. Pellet is suspended in 140 volumes of assaybuffer. Tissue is mixed with test compounds and after 10 min pre-incubation, 10 nM [³H]-noradrenaline is added to a final volume of 0.2 ml and the mixture is incubated for 15 min at 37° C. After 15 min incubation, samples are filtered directly on Unifilter GF/C glass fiber filters (soaked for 30 min in 0.1% polyethylenimine) under vacuum and immediately washed with 1×0.2 mL assay buffer. Non-specific uptake is determined using talsupram (10 μM final concentration). Duloxetine is included as reference in all experiments as dose-response curve.

### Measurements of [³H]Dopamine Uptake Into Rat Cortical Synaptosomes

Tissue preparation: male wistar rats (125-250 g) are sacrificed by decapitation and striatum quickly dissected out and placed in ice cold 0.40 M sucrose. The tissue is gently homogenised (glass teflon homogeniser) and the P2 fraction is obtained by centrifugation (1000 g, 10 minutes and 40000 g, 20 minutes, 4° C.) and suspended in 560 volumes of a modified Krebs-Ringer-phosphate buffer, pH 7.4.

Tissue 0.25 mg/well (140 μl) (original tissue) is mixed with test suspension. After 5 minutes pre-incubation at room temperature, 12.5 nM 3H-dopamine is added and the mixture is incubated for 5 minutes at room temperature. Final volume is 0.2 mL. The incubation is terminated by filtering the samples under vacuum through Whatman GF/C filters with a wash of 1×0.2 ml buffer. The filters are dried and appropriate scintillation fluid (Optiphase Supermix) is added. After storage for 2 hours in the dark the content of radioactivity is determined by liquid scintillation counting. Uptake is obtained by subtracting the non-specific binding and passive transport measured in the presence of 100 μM of benztropin. For determination of the inhibition of uptake ten concentrations of drugs covering 6 decades are used.

³H-DA=3,4-(ring-2,5,6-³H)dopamine hydrochloride from New England Nuclear, specific activity 30-50 Ci/mmol.

The following references are incorporated herein by reference in their entirety:

Hyttel, Biochem. Pharmacol. 1978, 27, 1063-1068;

Hyttel, Prog. Neuro-Psychopharmacol. & bil. Psychiat. 1982, 6, 277-295;

Hyttel & Larsen, Acta Pharmacol. Tox. 1985, 56, suppl. 1, 146-153.

As shown in Table 1, activity (IC₅₀) at the monoamine transporter for the compounds of the present invention was determined to be within the range of 0.1-200 nM.

TABLE 1
Compound Activity at the Monoamine Transporter

| | 5-HTT | DAT | NAT |
|---|---|---|---|
| | (IC₅₀ nM) | (IC₅₀ nM) | (IC₅₀ nM) |
| Formula VI | * | * | * |
| Formula VII | * | * | * |
| Formula VIII | * | * | * |
| Formula IX | * | * | * |
| Formula X | * | * | * |
| Indatraline | * | * | * |
| Sertraline | * | * | * |

* 0.1-200 nM.
5-HTT Serotonin Transporter
DAT Dopamine Transporter
NAT Noradrenergic Transporter

### In Vivo—Anti-Obesity Study

A compound of the present invention was tested to determine and compare its effects on body weight, food and water intake in the diet induced obese (DIO) male rats. Further, the compound was tested to determine whether it affected energy expenditure compared to vehicle treated rats as measured by indirect calorimetry (22 hour measurements). The study results also provide support for the basis that the compound can be effective for the treatment of related metabolic syndrome and diabetes such as diabetes Type 2.

a) Animals

Male Diet-Induced Obese (DIO) rats (Rattus norvegicus) selectively bred on a Sprague-Dawley background with a high propensity to develop diet induced obesity and insulin resistance upon exposure to a fat-rich high-caloric diet (Rheoscience A/S, Ledøje-Smørum, Denmark) were used in the study.

b) Experimental Description

A total of fifty (50) selectively bred male DIO rats were transferred from the breeding facilities to the test stables. The animals had reached the age of 26 weeks (22 weeks on high-fat diet). The rats were housed individually (1 rat/cage) under a controlled light cycle (light from 11:00 PM-11:00 PM) at controlled temperature and humidity conditions. The animals were offered an energy-dense high-fat diet (#12266B; Research Diets, New Brunswick, N.J.) and water ad libitum, unless otherwise stated.

Dosing, measurements of food intake, water intake and body-weight were performed in the morning unless otherwise stated.

From day 7, animals were handled on a daily basis to accustom them to the experimental procedures. On day 3, animals are mock gavaged on a daily basis. Food intake, water intake and body-weight were recorded daily from experimental day 3 to 6, twice weekly from experimental day 7 to 20 and once weekly from experimental day 21 to 49.

On day 1 the animals were stratified according to weight to participate in one of the treatment groups listed in Table C.

TABLE C
Dose Groups and Treatment

| | | DOSE | CONCEN- | DOSAGE | |
|---|---|---|---|---|---|
| GROUP | TEST | (MG/ | TRATION | VOLUME | NO. OF |
| NO. | ARTICLE | KG) | (MG/ML) | (ML/KG) | ANIMALS |
| 1 | Vehicle | 0 | N/A | PO: 5 | 10 |
| 2 | Compound | 0.75* | 0.15 | PO: 5 | 10 |
| 3 | Compound | 1.75* | 0.35 | PO: 5 | 10 |
| 4 | Compound | 3.5* | 0.7 | PO: 5 | 10 |
| 5 | Sibutramine⁺ | 5 | 1 | PO: 5 | 10 |

*Doses were decided on the basis of the pharmacokinetic profile results (see below).
⁺Sibutramine = sibutramine hydrochloride monohydrate (MERIDIA ®, Abbott Laboratories, N. Chicago, IL. USA).

First day of dosing was day 0. The compound solution was administered once daily at 10:00 AM (1 hour before lights off) on experimental days 0-20 by oral gavage using a gastric tube connected to a 3 ml syringe (Luer-Lok™, Becton-Dickinson, Franklin Lakes, N.J.). From experimental day 21, all groups entered a recovery period where the animals were not dosed.

From days 12-16 an indirect calorimetry test was performed on animals from groups 1, 4 and 5, according to the procedure described below.

On experimental day 21 (20 hours after the last dose) and 49, blood samples for pharmacokinetic profile were obtained on three randomly chosen animals from groups 1-3 according to the procedure below (Blood sampling—pharmacokinetic profile). Additionally blood samples for blood chemistry were obtained on all animals according to the procedure below (Blood sampling—blood chemistry). After blood sampling, two randomly chosen animals from groups 2-4 were killed under CO₂/O₂ anaesthesia by decapitation and blood samples collected according to the procedure below (Termination).

On experimental day 49 the study was terminated. Animals were killed under CO₂/O₂ anaesthesia by decapitation and blood samples collected according to the procedure below (Termination).

c) Pilot—Pharmacokinetic Profile

A total of six (6) selectively bred male DIO rats were transferred from the breeding facilities to the test stables. The animals had reached the age of 26 weeks (22 weeks on high-fat diet). The rats were housed individually (1 rat/cage) under a controlled light cycle (light from 11:00 PM-11:00 AM) at controlled temperature and humidity conditions. The animals were offered an energy-dense high-fat diet (#12266B; Research Diets) and water ad libitum, unless otherwise stated.

These animals were used for analyzing the pharmacokinetic profile of the compound of the present invention and the results were used to decide the doses that were tested in the main study. On the day of the experiment, animals were randomly divided into two groups having three (3) animals per group. Animals were dosed with the compound at 0.5 mg/kg or 2.5 mg/kg. Two hundred-forty (240) min after the administration of compound, animals were euthanized. To this effect, animals were anaesthetized with CO₂/O₂ and decapitated. Terminal blood was collected in ethylene diamine tetracetic acid (EDTA) coated tubes (4.9 ml blood, K₃-EDTA 1.6 mg/ml, Sarstedt AG & Co., Nümbrecht, Germany). The resulting plasma was transferred to non-coated tubes and stored at −80° C. Additionally brains were excised and snap frozen in crushed dry ice.

d) MR Scanning

Whole body composition was analyzed non-invasively by an EchoMRI 4-in-1-scanner (Echo Medical Systems, Houston, Tex., USA) on experimental days 1, 14 (just before indirect calorimetry), 21 and 49. The scanner assessed fat mass, lean tissue mass, free fluids, and total body water content. During the scanning procedure, the rat was placed in a restrainer for 90-120 seconds.

e) Energy Expenditure

Indirect calorimetry was performed at thermo-neutrality (29° C. in calorimetry cages; TSE Systems GmbH, Bad Homburg, Germany) on experimental days 12-16 on groups 1, 4 and 5—the remaining groups spent a similar time in the calorimetry room. The day before the test, eight (8) animals (randomly chosen across groups) were transferred to a temperature-controlled room (temperature set at 29° C.). The morning of the next day, the rats were weighed then transferred to air-tight plexiglass cages (no food and bedding, but water ad libitum). Airflow in and out was controlled by the calorimetry system. Oxygen consumption and CO₂ production was measured every 20 minutes over the next 22 hours (data from the first 1-hour of the test (acclimatization period) was excluded). Respiratory exchange ratio (RER; VCO2/VO2) and heat production (kcal/kg BW/h) was calculated for the light phase (10 hours) and the dark phase (12 hours). Following experimentation, rats were transferred to their home-cages and given free access to food and water.

f) Blood Sampling, Blood Chemistry

Blood samples were collected into pre-cooled tubes pre-coated with EDTA (350 μl blood, K₃-EDTA, 1.6 mg/mL, Sarstedt) for determination of liver enzymes alanine amino transferase (ALAT) and aspartate amino tranferase (ASAT) in plasma. The tubes were placed on wet ice pending processing. Blood samples were centrifuged at 4000×g, at 4° C., 15 min, the resulting plasma was transferred into non-coated tubes and stored at −80° C. until analyses. Blood samples were performed in the morning.

g) Blood Sampling (Pharmacokinetic Profile)

Blood samples were collected into pre-cooled tubes pre-coated with EDTA (200 μl blood, K₃-EDTA, 1.6 mg/mL, Sarstedt) for determination of the compound of the present invention in plasma. The tubes were placed on wet ice pending processing. Blood samples were centrifuged at 4000×g, at 4° C., 15 min, the resulting plasma was transferred into non-coated tubes and stored at −80° C. until analyses.

h) Termination

Animals were anesthetized with CO2/O2 and decapitated, and terminal blood samples were collected: 4.9 ml blood in EDTA tubes (K₃-EDTA 1.6 mg/ml, Sarstedt) and 4.0 ml blood in Li-heparin coated tubes (Li-Hep, Sarstedt). The blood was centrifuged at 4000×g, at 4° C., 15 min and plasma was separated in aliquots of 1 ml into non-coated tubes and stored at −80° C. Additionally, each brain was excised and snap frozen in crushed dry ice and stored at −80° C.

i) Analytical Methods

Liver enzymes, ALAT and ASAT, were measured in the collected blood samples (see above, Blood sampling—blood chemistry) using standard enzyme assay kits on a fully automated analyzer (Vitros DT 250 Chemistry System, The Johnson & Johnson Co., New Brunswick, N.J., USA).

j) Data Evaluation and Statistics

All data was subjected to relevant statistical analyses (GraphPad Prism vs. 5.01, GraphPad Software, Inc., La Jolla, Calif., USA). Results are presented as mean±SEM unless otherwise stated. Statistical evaluation of the data was carried out using one-way or two-way analysis of variance (ANOVA with Dunnet's post hoc test) with appropriate post-hoc analysis. Possible outliers are tested by Grubbs outlier-test before being excluded.

k) Results

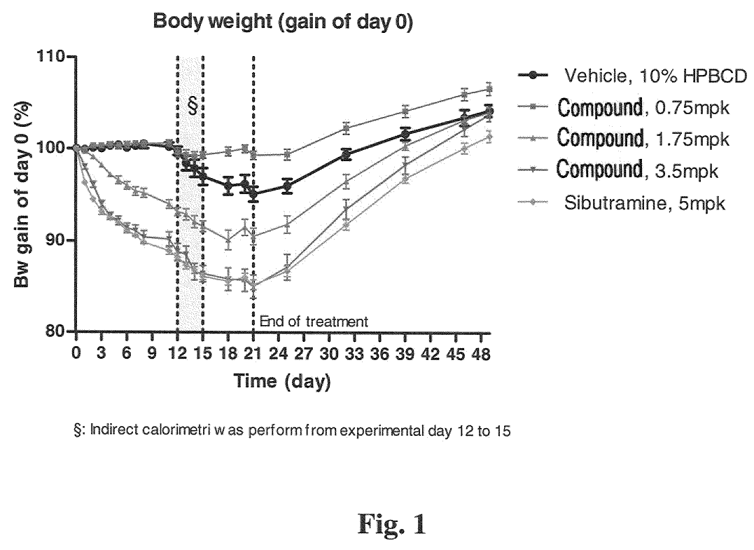

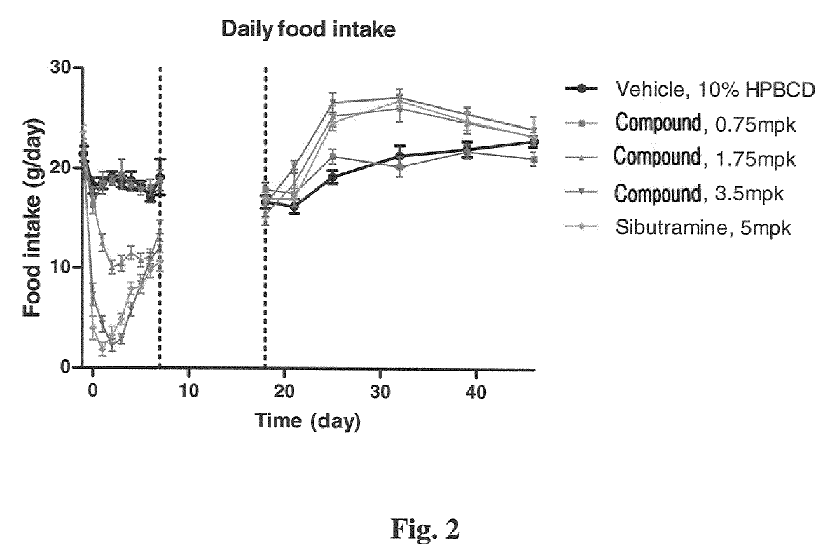

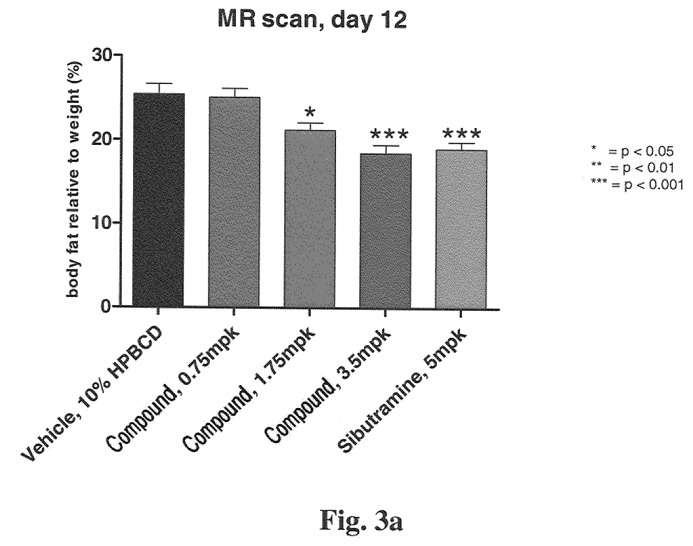

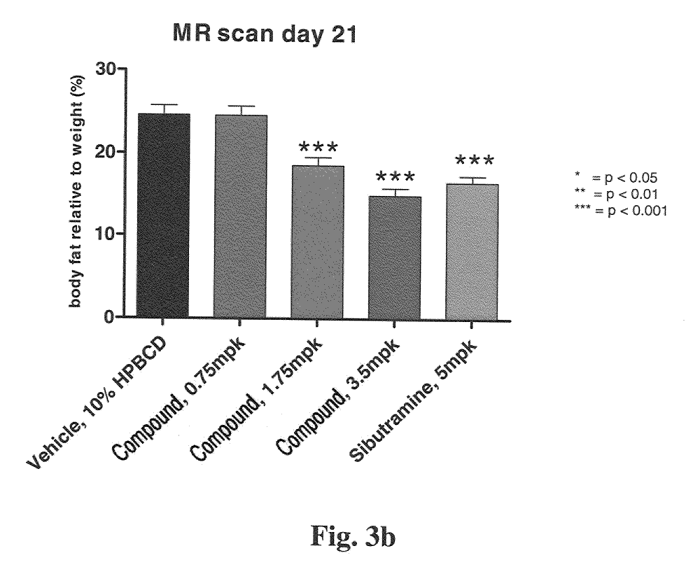

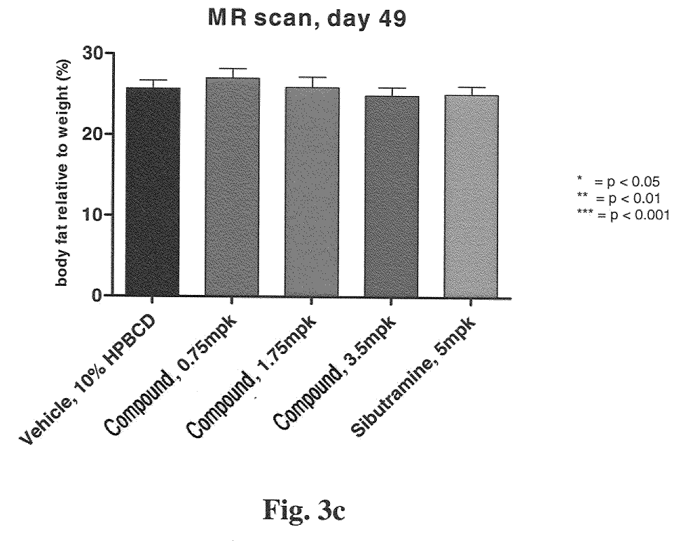

Treatment with the compound gave rise to a dose dependent lowering of body weight showing a clear decrease for the highest doses as compared to the vehicle controls. See FIG. 1. The loss of body weight was due to a significant lowering of daily food intake, where the food intake, comparable to the body weight data, was lowered in a dose dependent manner upon treatment with the compound. See FIG. 2. In addition to the food intake and body weight data, there was a significant dose dependent lowering of body fat content obvious at the scheduled 12 and 21 d timepoints, and reversed upon compound withdrawal. See FIGS. 3a-c. Further, the group treated with the highest dose of the compound (Group 4) showed similar effects on all parameters with the sibutramine group (Group 5). See FIGS. 1-3.

In summary, the compound showed an anorectic effect with a concomitant lowering of body weight. See FIGS. 1-2. The lowering of body weight was mainly caused by loss of body fat (white adipose tissue). See FIGS. 3a-c.

The study, therefore, showed that the compound may be effective for treatment of obesity, eating disorders, or a combination thereof.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention.

Each reference cited in the present application, including literature references, books, patents and patent applications, is incorporated herein by reference in its entirety

What is claimed is:

1. A method of treating obesity or an eating disorder, comprising administering to a patient in need thereof a therapeutically effective amount of a compound represented by Formula I: 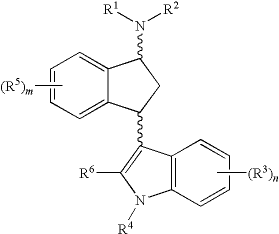
wherein:
each R¹ and R² is independently hydrogen, C₁-C₈-straight or branched alkyl or C₃-C₈-cycloalkyl; or wherein R¹ and R² and the nitrogen to which they are attached form azetidine, piperidine, pyrrolidine, azapane or morpholine;
each R³ is independently hydrogen, C₁-C₈-straight or branched alkyl, C₁-C₅-alkoxy, C₁-C₈-straight or branched polyfluoroalkyl, halogen, cyano, hydroxyl, tetrazole-optionally substituted with methyl, or amino; or two R³ groups on adjacent carbons combine together to form a methylenedioxy linker;
R⁴ is hydrogen, C₁-C₈-straight or branched alkyl or C₃-C₈-cycloalkyl;
each R⁵ is hydrogen, halogen C₁-C₅-alkoxy, C₁-C₈-straight or branched alkyl, C₁-C₈-straight or branched polyfluoroalkyl, cyano, or hydroxyl;
m is an integer from 1 to 4 inclusive;
n is an integer from 1 to 4 inclusive; and
R⁶ is hydrogen, C₁-C₈-straight or branched alkyl or phenyl;
or pharmaceutically acceptable salts thereof.

2. The method of claim 1, wherein the compound is a cis isomer.

3. The method of claim 1, wherein the compound is a trans isomer.

4. The method according to claim 1, wherein the compound represented by Formula I is selected from the group consisting of Formula II-V:

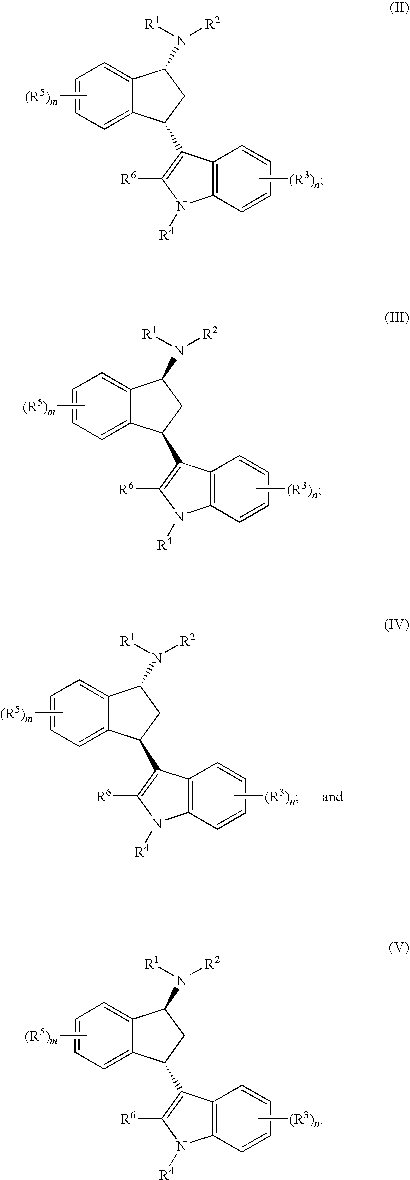

5. The method of claim 4, wherein R¹ is hydrogen.

6. The method of claim 5, wherein R² is methyl.

7. The method of claim 6, wherein R⁴ is hydrogen.

8. The method of claim 7, wherein R³ is selected form the group consisting of hydrogen, halogen, and methoxy.

9. The method of claim 8, wherein R³ is a hydrogen or halogen.

10. The method of claim 9, wherein the halogen is fluorine or chlorine.

11. The method of claim 10, wherein R⁵ and R⁶ are hydrogen.

12. The method of claim 1, wherein the eating disorder is selected from the group consisting of bulimia nervosa, anorexia nervosa and binge eating disorder.

13. A method of treating obesity or an eating disorder, comprising administering to a patient in need thereof a therapeutically effective amount of a compound selected from the group consisting of: 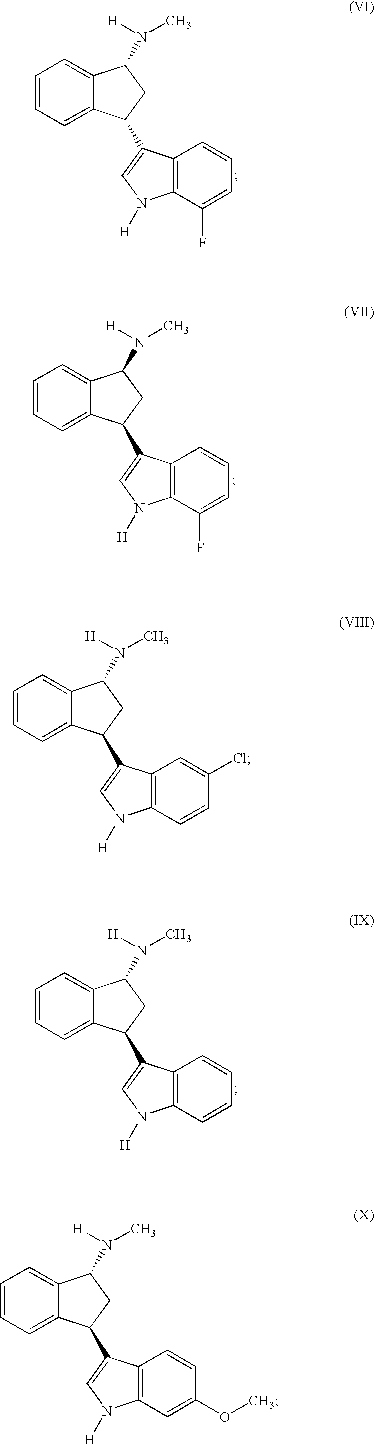 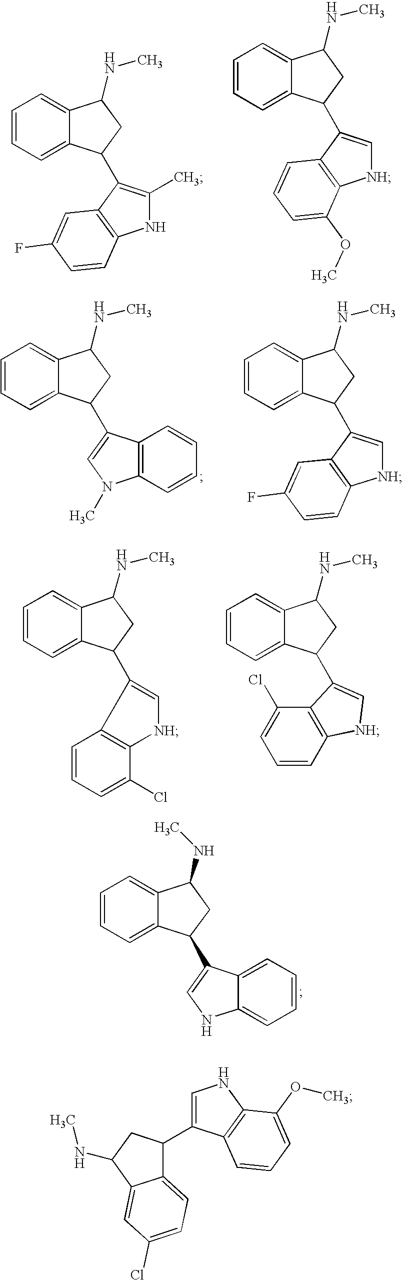 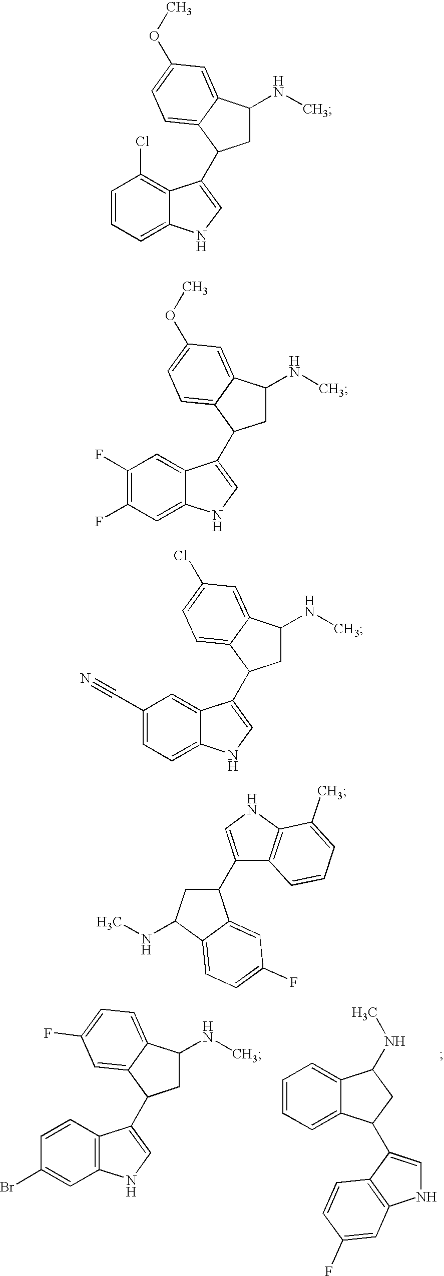 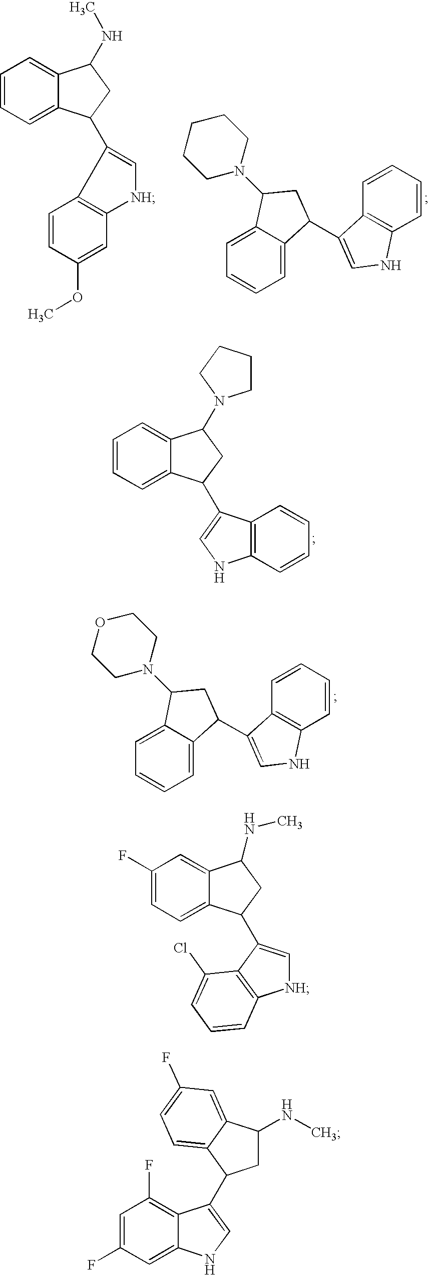 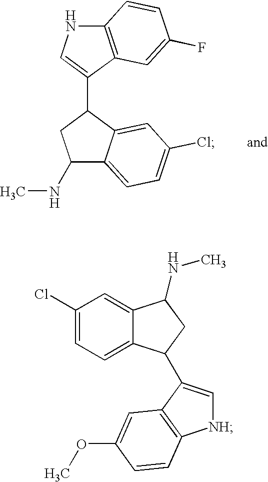
or a pharmaceutically acceptable salt thereof.

14. The method of claim 13, wherein the eating disorder is selected from the group consisting of bulimia nervosa, anorexia nervosa and binge eating disorder.
